(19) United States
(12) Patent Application Publication
(10) Pub. No.: US 2024/0081971 A1
(43) Pub. Date: Mar. 14, 2024

(54) SELF-EXPANDING TISSUE LUMEN STENTS WITH DRAINAGE ENHANCEMENT FEATURES

(71) Applicant: BOSTON SCIENTIFIC SCIMED, INC., Maple Grove, MN (US)

(72) Inventor: Martyn G. Folan, Galway (IE)

(73) Assignee: BOSTON SCIENTIFIC SCIMED, INC., Maple Grove, MN (US)

(21) Appl. No.: 18/464,896

(22) Filed: Sep. 11, 2023

Related U.S. Application Data
(60) Provisional application No. 63/405,828, filed on Sep. 12, 2022.

(51) Int. Cl. A61F 2/04

(52) U.S. Cl. CPC A61F 2/04; A61F 2002/0081

(57) ABSTRACT

The disclosure provides a tissue lumen stent that has an elongated tubular configuration and a foreshortened configuration in which the upstream, the downstream, or both the upstream and downstream ends expand radially into flanged and/or flared structures while the region therebetween is generally cylindrical. The upstream flange structure has a larger maximum lateral dimension, axial width and/or axial radius than that of the downstream flange structure and may include an inclined portion having an axial length at least as long as a maximum diameter of the saddle region when the body is in the foreshortened configuration.

### CROSS-REFERENCE TO RELATED APPLICATIONS

This application claims the benefit of U.S. Provisional Patent Application Ser. No. 63/405,828 filed on Sep. 12, 2022, the disclosure of which is incorporated herein by reference.

### FIELD

This application relates generally to medical methods and devices. More specifically, the present disclosure relates to lumen stents and methods for their use in maintained lumen patency with medical procedures.

### BACKGROUND

Tissue lumen stents are often used in medical procedures to maintain lumen patency. Typically, the tissue lumen stent has a body with upstream and downstream ends and a central region therebetween. Medical procedures can be provided, which include (a) accessing a biliary system of a patient with an endoscope, and (b) deploying, within the biliary system of the patient, a tissue lumen stent such that the tissue lumen stent contacts a lumen in the biliary system of the patient, such as, for example, the common bile duct, the pancreatic duct, or the hepatic duct.

### BRIEF SUMMARY

Various embodiments described herein provide a tissue lumen stent with features to improve, enhance, or facilitate drainage. In general, the tissue lumen stents have an elongated tubular configuration and a foreshortened configuration in which the upstream, the downstream, or both the upstream and downstream ends expand radially into flanged and/or flared structures while the region therebetween is generally cylindrical.

In some cases, when the stent is in the foreshortened configuration, the upstream flange structure has a larger maximum lateral dimension, axial width and/or axial radius than that of the downstream flange structure and may include an inclined portion having an axial length at least if a maximum diameter of the saddle region when the body is in the foreshortened configuration. On the other hand, some embodiments are characterized by a downstream flange structure that has a larger maximum lateral dimension, axial width and/or axial radius than that of the upstream flange structure. Alternatively, or additionally, the upstream flange structure can include a distal-most opening having a diameter larger than a maximum internal diameter of the saddle region when the body is in the foreshortened configuration. In certain embodiments, the body includes a covered mesh, and in some cases, may comprise both covered and uncovered mesh, while some embodiments include a covering or membrane over at least the cylindrical saddle portion of the stent and, optionally, one or both upstream and downstream flange structures.

In some implementations, the present disclosure be embodied as a stent, for example, a stent comprising a body comprising an elongated tubular configuration and a foreshortened configuration, wherein an upstream end of the body expands into an upstream structure and the downstream end of the body expands into a flange structure when in the elongated tubular configuration, the body comprising a cylindrical saddle region disposed between the upstream structure and the flange structure, the body comprising a channel running helically around the body, the channel being defined on the exterior of the body and reflected on the interior of the body.

Alternatively, or additionally in any of the embodiments of a stent above the channel has a consistent pitch along the longitudinal length of the body.

Alternatively, or additionally in any of the embodiments of a stent above a pitch of the channel can increase along a longitudinal length of the body.

Alternatively, or additionally in any of the embodiments of a stent above the pitch increases along the longitudinal length of the body from the upstream end to the downstream end.

Alternatively, or additionally in any of the embodiments of a stent above a width of the channel can be wider at the upstream end of the body than at the downstream end of the body.

Alternatively, or additionally in any of the embodiments of a stent above a width of the channel can be wider at the downstream end of the body than at the upstream end of the body.

Alternatively, or additionally in any of the embodiments of a stent above the upstream structure comprises a flange or a flare.

In some implementations, the present disclosure be embodied as a stent, for example, a stent comprising a body comprising an elongated tubular configuration and a foreshortened configuration, wherein an upstream end of the body expands into an upstream structure and the downstream end of the body expands into a flange structure when in the elongated tubular configuration, the body comprising a cylindrical saddle region disposed between the upstream structure and the flange structure, the body comprising a coating over the downstream end of the body and the cylindrical saddle region, the upstream structure comprising a plurality of anti-migration fins disposed on an external surface.

Alternatively, or additionally in any of the embodiments of a stent above the plurality of anti-migration fins comprising wire fins arranged to flare away from the upstream structure.

Alternatively, or additionally in any of the embodiments of a stent above the plurality of anti-migration fins comprising a point facing towards the downstream end of the body.

Alternatively, or additionally in any of the embodiments of a stent above the at least one of the plurality of anti-migration fins can comprise a point facing towards the downstream end of the body and at least one other one of the plurality of anti-migration fins can comprise a point facing towards the upstream end of the body.

Alternatively, or additionally in any of the embodiments of a stent above the upstream structure can comprise a flare.

In some implementations, the present disclosure be embodied as a stent, for example, a stent, comprising a body comprising an elongated tubular configuration and a foreshortened configuration, wherein an upstream end of the body expands into an upstream flange structure and the downstream end of the body expands into a downstream flange structure when in the elongated tubular configuration, the body comprising a cylindrical saddle region disposed between the upstream structure and the flange structure, the cylindrical saddle region comprising a curve along a longitudinal direction of the body.

Alternatively, or additionally in any of the embodiments of a stent above the cylindrical saddle region can comprise another curve along an axial direction of the body.

Alternatively, or additionally in any of the embodiments of a stent above the curve is closer to the upstream flange structure than the downstream flange structure or closer to the downstream flange structure than the upstream flange structure.

In some implementations, the present disclosure be embodied as a method, for example, a method of treating a patient, comprising accessing a biliary system of a patient with an endoscope; and deploying, within the biliary system of the patient, a stent, the stent having a body comprising an elongated tubular configuration and a foreshortened configuration, wherein an upstream end of the body expands into an upstream structure and the downstream end of the body expands into a flange structure when in the elongated tubular configuration, the body comprising a cylindrical saddle region disposed between the upstream structure and the flange structure, the body comprising a channel running helically around the body, the channel being defined on the exterior of the body and reflected on the interior of the body.

Alternatively, or additionally in any of the embodiments of a method above the channel can have a consistent pitch along the longitudinal length of the body.

Alternatively, or additionally in any of the embodiments of a method above a pitch of the channel can increase along a longitudinal length of the body.

Alternatively, or additionally in any of the embodiments of a method above the pitch increases along the longitudinal length of the body from the upstream end to the downstream end.

Alternatively, or additionally in any of the embodiments of a method above a width of the channel is wider at the upstream end of the body than at the downstream end of the body.

### BRIEF DESCRIPTION OF THE SEVERAL VIEWS OF THE DRAWINGS

To easily identify the discussion of any element or act, the most significant digit or digits in a reference number refer to the figure number in which that element is first introduced.

FIG. 1A illustrates a biliary system.

FIG. 1B illustrates the biliary system of FIG. 1A with a stent 120 placed therein.

FIG. 2A illustrates a stent 200, in accordance with at least one embodiment of the present disclosure.

FIG. 2B illustrates the stent 200, in alternative detail.

FIG. 3 illustrates a stent 300, in accordance with at least one embodiment of the present disclosure.

FIG. 4A illustrates a stent 400a, in accordance with at least one embodiment of the present disclosure.

FIG. 4B illustrates a stent 400b, in accordance with at least one embodiment of the present disclosure.

FIG. 5A illustrates a stent 500a, in accordance with at least one embodiment of the present disclosure.

FIG. 5B illustrates a stent 500b, in accordance with at least one embodiment of the present disclosure.

FIG. 6A illustrates a stent 600a, in accordance with at least one embodiment of the present disclosure.

FIG. 6B illustrates a stent 600b, in accordance with at least one embodiment of the present disclosure.

FIG. 7 illustrates a biliary system of a patent with a stent placed therein.

FIG. 8A illustrates a stent 800a, in accordance with at least one embodiment of the present disclosure.

FIG. 8B illustrates a stent 800b, in accordance with at least one embodiment of the present disclosure.

FIG. 8C illustrates a stent 800c, in accordance with at least one embodiment of the present disclosure.

FIG. 8D illustrates a stent 800b, in accordance with at least one embodiment of the present disclosure.

FIG. 9A illustrates a biliary system of a patient.

FIG. 9B illustrates the biliary system of FIG. 9A in alternative detail.

### DETAILED DESCRIPTION

The present disclosure uses the terms anterograde, retrograde, downstream, upstream, proximal, distal, lower, upper, inferior and superior to refer to various directions. Unless the context clearly indicates otherwise, the terms anterograde, downstream, proximal, lower and inferior will generally be used synonymously to indicate a direction that is in line with fluid flow and along the devices and instruments toward the surgeon. Conversely, the terms retrograde, upstream, distal, upper and superior will generally be used synonymously to indicate a direction that is against fluid flow and along the devices and instruments away from the surgeon. It should be noted, however, that this nomenclature is being defined here to help clarify the following descriptions rather than to limit the scope of the invention. While the exemplary embodiments disclosed herein focus on entry and placement in a retrograde direction, the disclosed methods, systems and devices may in some circumstances be placed in an anterograde direction. In such situations, the “upstream” and “downstream” designations may be reversed.

As introduced above, the present disclosure described expandable stents used in the biliary system of a patient. As such a discussion of the biliary system is provided here. Bile, required for the digestion of food, is excreted by the liver into passages that carry the bile into the left hepatic duct 102 and the right hepatic duct 104. These two hepatic ducts merge to form the common hepatic duct 106. The common hepatic duct 106 exits the liver and joins the cystic duct 108 from the gallbladder 110, which stores bile, to form the common bile duct 112. The common bile duct 112, in turn, joins with the pancreatic duct 114 from the pancreas to feed bile, pancreatic juice and insulin into the descending part of the duodenum 116 through the ampulla of Vater 118. A sphincter, known as the sphincter of Oddi, is located at the opening of the ampulla of Vater 118 into the duodenum 116 to prevent matter in the duodenum 116 from traveling in a retrograde direction up into the common bile duct 112.

Tumor growth, hyperplasia, pancreatitis or other strictures in or around the biliary duct tree outlined above can impede or block the flow of fluid from the liver, gallbladder and/or pancreas to the duodenum. To alleviate the effects of the stricture, a stent may need to be placed in a portion of the biliary system. The stent may be placed endoscopically. One procedure for placing the stent is endoscopic retrograde cholangiopancreatography (ERCP). ERCP is a technique that combines the use of endoscopy and fluoroscopy to diagnose and treat certain problems of the biliary or pancreatic ductal systems. The procedure involves placing an endoscope down the esophagus, through the stomach, into the duodenum, then passing various accessories through the endoscope instrumentation channel up through the ampulla of Vater into the biliary or pancreatic ductal systems. Alternatively, a special slim-diameter endoscope, sometimes referred to as a peroral cholangioscope, may be passed directly into the bile or pancreatic ducts.

Thus, stents currently placed by ERCP are used to facilitate drainage of bile through the biliary tree. Drainage is a commonly expressed desire for self-expanding stents, allowing residual drainage from secondary sources. The present disclosure describes and depicts several self-expanding stents with improved drainage features or characteristics.

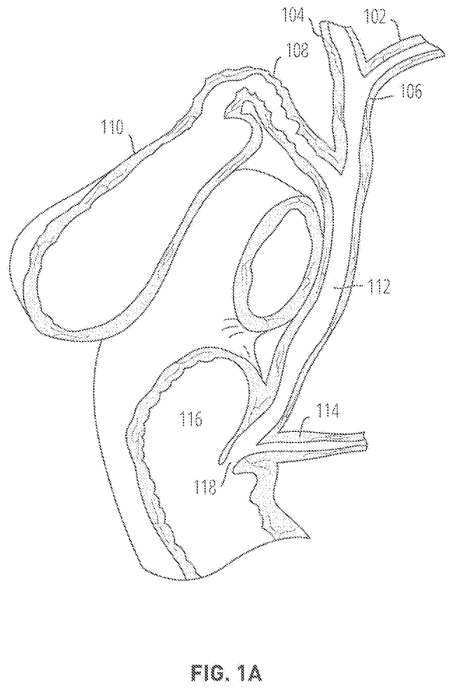

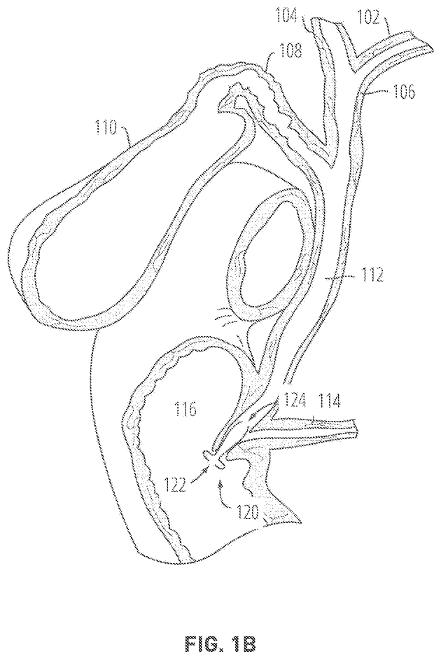

FIG. 1B illustrates an exemplary biliary stent 120 implanted in the lower end of the common bile duct 112. In such a configuration, stent 120 may be used to treat an ampullary stenosis. In other embodiments, the stent 120 may be longer to bridge a bile duct stricture higher upstream. Stent 120 comprises a downstream end 122 that protrudes into the duodenum 116, and an upstream end 124 that extends up into the common bile duct 112. Stent 120 is shown in a generally radially expanded and axially foreshortened state, such that it is contacting the walls of the common bile duct 112 continuously along its length, or at least in several places.

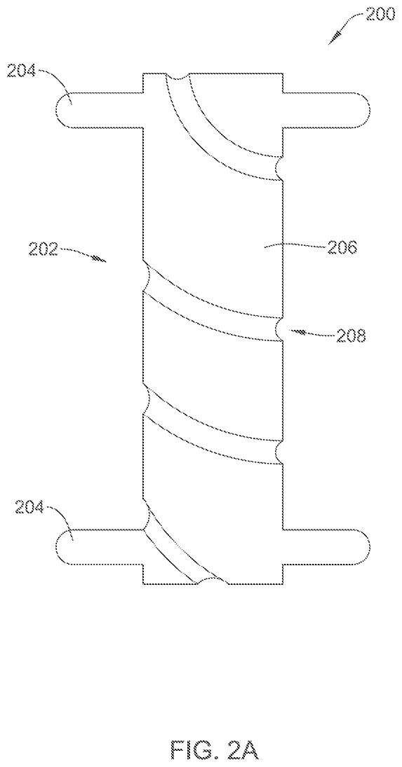

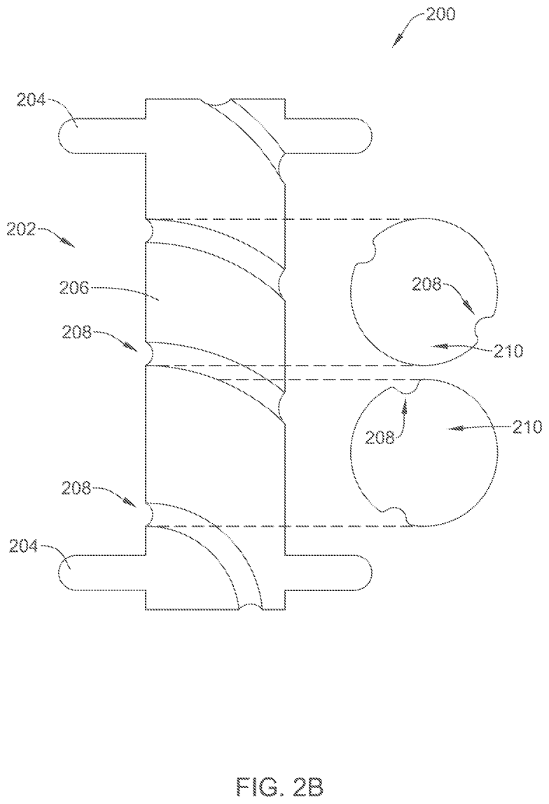

FIG. 2A illustrates a stent 200, according to some embodiments of the present disclosure. The stent 200 includes a body 202 having a generally tubular formation. The body 202 can be formed from a woven filament braid. The filament will typically be a metal wire, more typically being a nickel-titanium or other super-elastic or shape memory metal wire. Alternatively, in cases where elasticity is less critical, a filament could be formed from a polymeric material, such as polypropylene, polyethylene, polyester, nylon, PTFE, or the like. In some cases, a bioabsorbable or bio-degradable material, typically a biodegradable polymer, such as poly-L-lactic acid (PLLA), could be used.

The body 202 may have both an elongated tubular configuration (for delivery of the stent) and a foreshortened configuration (when deployed) where downstream and upstream ends of the body expand radially (as the body is foreshortened). One or both ends of the body 202 may expand into flanges 204 (e.g., double-walled flange structures, or the like). Such “double-walled flange structures” may be formed as a portion of the body, typically an end-most portion but optionally some portion spaced inwardly from the end, moves inwardly (toward the middle) so that a pair of adjacent body segments within the portion are drawn together at their bases so that a midline or a crest line bends and expands radially to form a pair of adjacent annular rings which defines the flanges 204 having the double-walled flange structure. After such foreshortening and deployment of the double-walled flange structures, the body 202 may further have a cylindrical saddle region 206 formed between the flanges 204.

Further, the body 202 can have a channel 208 running in a helical pattern around the body 202 and the cylindrical saddle region 206. The channel 208 can run the length of the stent 200. It is noted that FIG. 2A illustrates a view of the outside of stent 200 while FIG. 2B illustrates a cut away view at two points along a longitudinal length of the stent 200 showing the inner lumen 210 of stent 200. As can be seen from these figures, the channel 208 represented on the external surface of the stent 200 translate or transfer to the inner lumen 210. As described herein, stent 200 can be formed as a single walled braided device. As such the channel 208 running the length of the stent 200 will be represented on both the external and internal surfaces of the body 202, thereby encouraging flow of bile (or other material) through the stent 200. As depicted, the channel 208 can be defined from the distal end of the stent 200 to the proximal end of the stent 200.

The stent 200, when formed from shaped memory metal wires, such as nitinol or eligiloy, the wires may have a relatively small diameter, typically in the range from 0.001 inches to 0.02 inches, usually from 0.002 inches to 0.01 inches, where the braid may include from as few as 10 to as many as 200 wires, more commonly being from 20 wires to 200 wires. In exemplary cases, the wires will be round having diameters in the range from 0.003 into the 0.007 inches with a total of from 24 to 60 wires. The wires may be braided into a tubular geometry by conventional techniques, and the tubular geometry may be heat-treated to impart the desired shape memory. Usually, the braided tube will be formed into the desired final (e.g., deployed) configuration with the flanges at each end. Such a flanged configuration may then be heat set or formed into the braid so that, in the absence of a radially constraining or axially elongating force, the stent will assume the foreshortened configuration with the flanges at each end. Such foreshortened-memory configurations allow the stent to be delivered in a constrained configuration (e.g., either radially or axially elongated) and thereafter released from constraint so that the body 202 assumes the flanged configuration (e.g., flanges 204) at the target site.

In alternative embodiments, however, the woven filament braid may be heat set into the elongated tubular configuration and shifted into the foreshortened, flanged configuration by applying an axial compressive force. Such axial compression will foreshorten and radially expand the flanges and allow a controlled and adjustable foreshortening, allowing the stent to be adjusted to a desired length. The woven filament braid, according to this embodiment, can be heat set to the expanded configuration and include a mechanism to mechanically foreshorten the stent beyond its normal fully expanded configuration, allowing the stent to automatically or manually adjust to the length of the stricture. The foreshortening and flanges may be formed by providing sleeves, tubes, rods, filaments, tethers, springs, elastic members or the like, which apply spontaneous or applied force to the tube to create foreshortening and flange formation. Optionally or additionally, the body 202 may have weakened regions, reinforced regions, or be otherwise modified so that the desired flange geometries are formed when a force is applied to cause axial foreshortening.

The stents described herein (e.g., stent 200) may be adapted to be delivered by a delivery device, typically an endoscopic delivery catheter, usually having a small diameter in the range from 1 mm to 8 mm, usually from 2 mm to 5 mm. Thus, the elongated tubular configuration of the body 202 will usually have a diameter less than that of the catheter diameter, usually from 0.8 mm to 7.5 mm, more usually from 0.8 mm to 4.5 mm, where the flange structures will be expandable significantly, usually being in the range from 3 mm to 70 mm, more usually in the range from 5 mm to 40 mm. A variety of stents having different lengths may be provided, in kit form for example, for use on strictures in different locations. In some embodiments, the overall lengths of the stents in their fully expanded/deployed state are 7, 9 and 11 cm. In other embodiments, the lengths are 6, 8 and 10 cm. In yet other embodiments, the stents will have lengths between 1 and 6 cm. The cylindrical saddle region 206 of the stent 200 will often not increase in diameter during deployment, but may optionally increase to a diameter from 2 mm to 50 mm, more usually from 5 mm to 12 mm. When present, the lumen or passage through the deployed stent 200 can have a variety of diameters, typically from as small as 0.2 mm to as large as 40 mm, more usually being in the range from 1 mm to 20 mm, and typically having a diameter which is slightly smaller than the expanded outside diameter of the cylindrical saddle region 206. The length of the body may also vary significantly. Typically, when in the elongated tubular configuration, the body will have a length in the range from 7 mm to 200 mm, usually from 12 mm to 70 mm. When deployed, the body 202 may be foreshortened, typically by at least 20%, more typically by at least 40% and often by 70% or greater. Thus, the foreshortened length will typically be in the range from 2 mm to 80 mm, usually in the range from 30 mm to 60 mm.

The body 202 of the stent 200 may consist of the woven filament braid with no other coverings or layers. In other instances, however, the stent 200 may further comprise a membrane or other covering formed over at least a portion of the body 202. Often, the membrane is intended to prevent or inhibit tissue ingrowth to allow the device to be removed after having been implanted for weeks, months, or longer. Suitable membrane materials include polytetrafluoroethylene (PTFE), expanded PTFE (ePTFE), silicone, polypropylene, urethane polyether block amides (PEBA), polyethyleneterephthalate (PET), polyethylene, C-Flex® thermoplastic elastomer, Krator® SEBS and SBS polymers, and the like.

Such membranes may be formed over the entire portion of the body 202 of the stent 200 or only a portion thereof, may be formed over the exterior or interior of the body 202, and will typically be elastomeric so that the membrane conforms to the body 202 in both the elongated and foreshortened configurations. Optionally, the membrane may be formed over only the central portion of the cylindrical saddle region 206, in which case the membrane would not have to be elastomeric when the central portion of the cylindrical saddle region 206 does not radially expand.

The covering or membrane inhibits tissue ingrowth within the interstices of the wire mesh and minimizes fluid leakage when the stent is implanted. Reducing tissue ingrowth improves the removability of the stents. In contrast to vascular stents, which are typically not designed to be moved or retrieved, the stents illustrated herein are collapsible and designed to be removable and retrievable. The stents also typically do not include barbs or other sharp projections used in some other types of stents to permanently secure the stent to surrounding tissue.

Different parts of the stent can be covered or uncovered depending on the specific application. In some embodiments, one end of the stent can have an uncovered portion. In some embodiments, any of the stents disclosed herein can include a covering on one of the ends of the stent. The covering can be on a flanged end of the stent or an end of the stent without a flange. For example, if deploying one end of the stent in the liver and the other end in the stomach then the end of the stent within the liver could be uncovered with the cylindrical saddle region 206 and end interfacing the stomach covered. If deploying one end adjacent to the ampulla of Vater and duodenum and the other end in the bile duct than the bile duct end would be covered. In some embodiments, any of the stents disclosed herein can include a covering on both ends of the stent. In some embodiments, a middle portion or portion between the upstream and downstream flanges can be uncovered. An uncovered middle portion can be used to drain fluid from the pancreatic duct when the ends of the stent are placed in the duodenum and bile duct.

In some embodiments, the cylindrical saddle region 206 is covered to prevent fluid from leaking outside of the cylindrical saddle region 206 of the stent 200. The stents disclosed herein can be deployed within the body such that the cylindrical saddle region 206 forms a fluid conduit between the body lumens in the peritoneum as described herein. The covered cylindrical saddle region 206 can prevent leakage into the peritoneum. Leaking biological material into the peritoneum can cause serious complications, as a result the stents can have a covering to prevent fluid or material leaking outside of the cylindrical saddle region 206 of the stent 200. Coverings can also be used on the end of the stent that is configured to connect to the stomach or duodenum.

Examples of manufacturing techniques that can be used to produce the stents disclosed herein include using laser cutting, weaving, welding, etching, and wire forming. A membrane material such as silicon can be applied to the wire stent frame to prevent the passage of fluid through the stent walls. The membrane or covering material can be applied by painting, brushing, spraying, dipping, or molding.

Further, in some embodiments, stent 200 can be formed by weaving a wire (or wires) on a mandrel and then clamping a sleeve over the mandrel to form the stent 200. In particular, the mandrel can have a mirror of the channel 208 inscribed into it and a matching sleeve with a protruding channel 208 feature that fits into the mandrel can create the channel 208 on the body 202 of the stent 200. Further, the formed stent can be annealed using an annealing process.

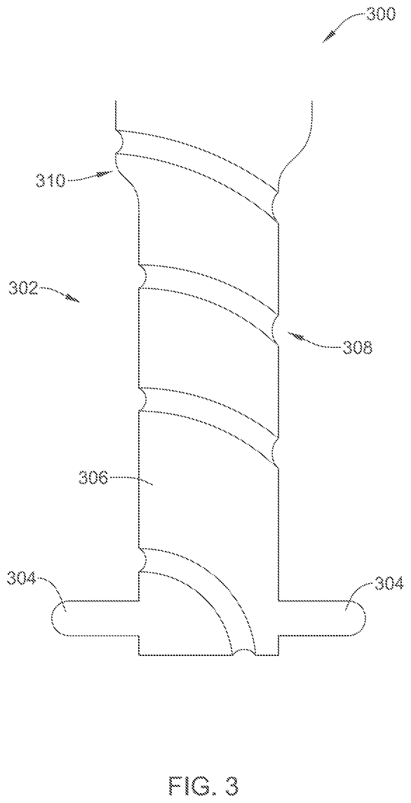

FIG. 3 illustrates a stent 300, according to some embodiments of the present disclosure. The stent 300 can have a body 302, flange 304, cylindrical saddle region 306, and channel 308 like the stent 200 of FIG. 2A and FIG. 2B. However, stent 300 may have flanges 204 at one end and a flare 310 at the opposite end. In general, the flare 310 is configured to prevent or inhibit downstream migration of stent 300.

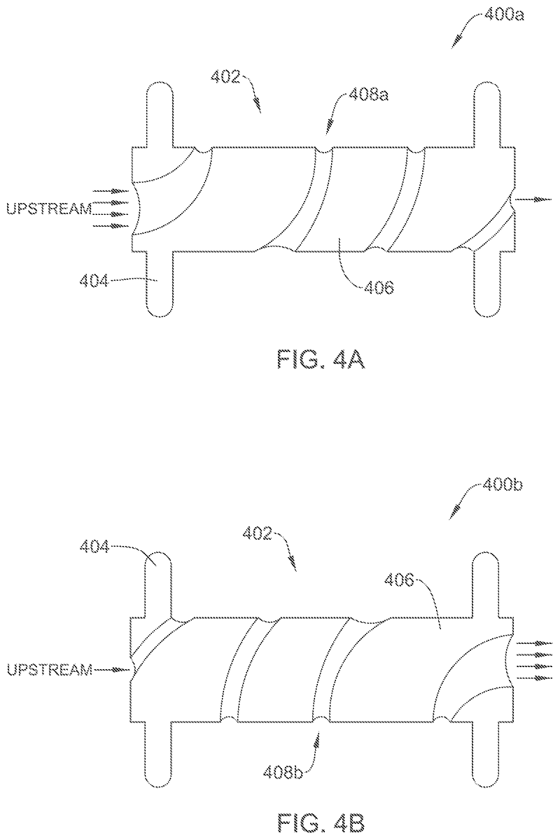

FIG. 4A and FIG. 4B illustrate stent 400a and stent 400b, respectively, according to some embodiments of the present disclosure. The stents 400a and 400b shown in these figures have a body 402, flange 404, cylindrical saddle region 406 and channels 408a and 408b. However, the helical channels 408a and 408b are not symmetrical. For example, FIG. 4A illustrates stent 400a having channel 408a, which is wider at the upstream end of the stent 400a and narrows as the channel 408a runs (or winds) along the longitudinal length of the stent 400a.

Similarly, FIG. 4B illustrates stent 400b having channel 408b, which is narrower at the upstream end of the stent 400b. The channel 408b widens as the channel 408b runs (or winds) along the longitudinal length of the stent 400b. In some applications, multiple inputs to the helical channels can provide a pathway for fluids to run down the length of the stents such that the combined volume may overpower the channel, causing backup and channel blockage. However, the nonsymmetrical channels as depicted in these figures may overcome this limitation. Similarly, a wider inlet channel (e.g., channel 408a) may provide a greater opportunity to align with the side branches and act as a funnel to direct the fluids down the helical channel. Also, the wider inlet may act as a reservoir, providing a downward force on the volume, encouraging continuous flow through the channel.

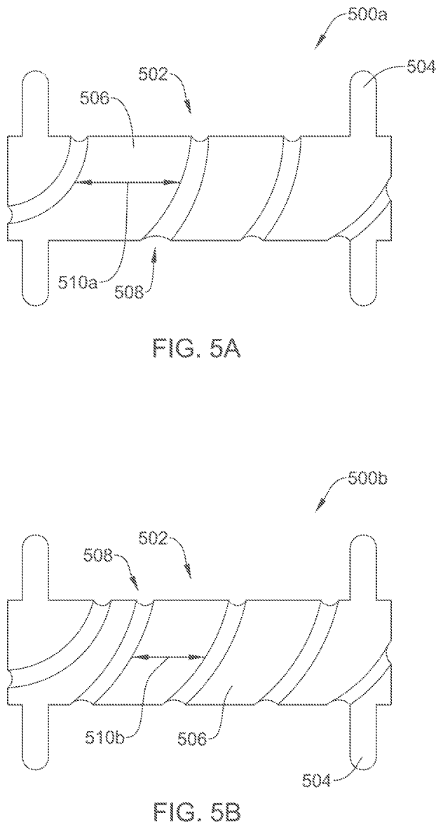

FIG. 5A and FIG. 5B illustrate stent 500a and stent 500b, respectively, according to some embodiments of the present disclosure. The stents 500a and 500b shown in these figures have a body 502, flange 504, cylindrical saddle region 506 and channels 508 having different pitches 510a and 510b. More particularly, the helical channels 508 have different spacing or distance between consecutive channels 508. For example, FIG. 5A illustrates stent 500a having channel 508 with pitch 510a, which is wider than the pitch 510b of channel 508 of stent 500b shown in FIG. 5B. In some examples, stents 500a and 500b can be intended for deployment in different locations based on the density (or pitch) of channel 508.

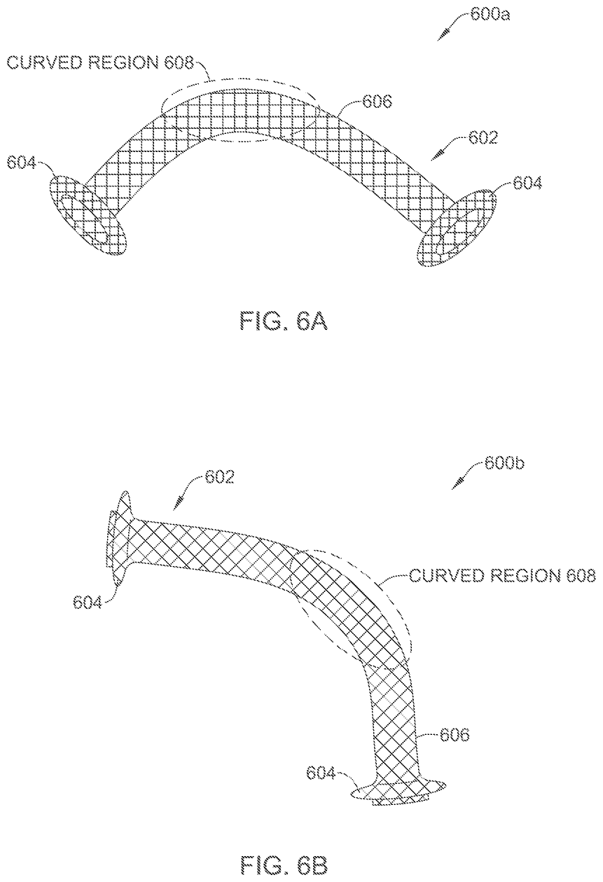

FIG. 6A and FIG. 6B illustrate stents 600a and 600b, respectively, according to some embodiments of the present disclosure. In general, the stents 600a and 600b can be like the Axios® stents available from Boston Scientific® and may be configured to be delivered via a Hot Axios™ device. For example, the stents 600a and 600b can have a body 602 and flanges 604 as well as cylindrical saddle region 606. Of note, however, stents 600a and 600b have a curved region 608 in the cylindrical saddle region 606. The curved region 608 can be curved in the longitudinal direction of the stents.

In some embodiments, the stents 600a and 600b can be used for the management of symptomatic cholecystitis in patients who are at high risk or unsuitable for surgery. It is to be appreciated that early laparoscopic cholecystectomy is considered, in most cases, the treatment of choice for acute cholecystitis. However, in the elderly, in critically ill patients, and in those with significant comorbidities, cholecystectomy is considered a high-risk procedure, and gallbladder drainage (GBD) is recommended as an alternative treatment.

Until now, percutaneous transhepatic gallbladder drainage (PTGBD) has been the most common GBD technique used in clinical practice. Even though the technical success rate of PTGBD is high at 98.9%, clinical success is lower at 86.0%, with adverse events such as intrahepatic hemorrhage, pneumothorax, biliary peritonitis, and pneumonia contributing to a procedure mortality rate of 4.0%. With readmission rates as high as 42% and reoccurrence between 4.1 and 22%, additional treatment options are required to complement existing management strategies.

The present stents 600a and 600b, when deployed via a system like the Hot Axios™ system, can be an option for patients at high risk or unsuitable for surgery. Published literature has demonstrated clinical and technical success for symptomatic cholecystitis in patients at high risk or unsuitable for surgery by creating a new temporary opening between the gallbladder and GI tract (e.g., duodenal location). EUS-GBD using Hot Axios™ is an option in high-risk surgical patients with acute cholecystitis when performed by an experienced endoscopist.

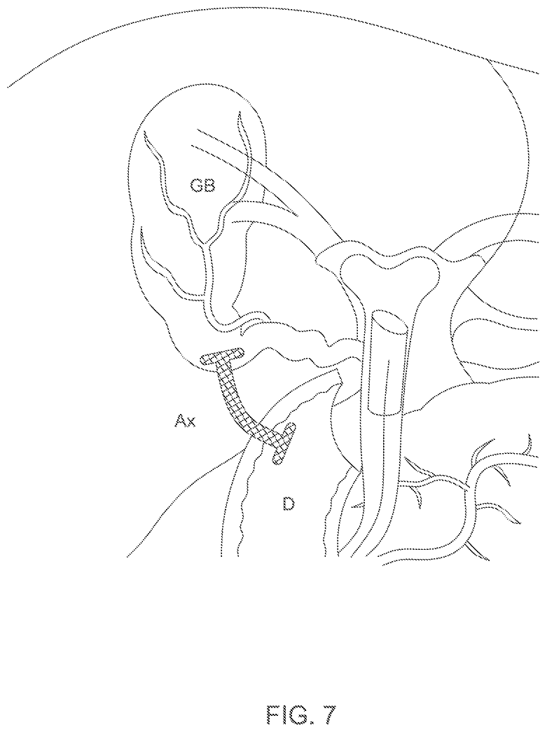

However, there is some potential risk for potential food impaction from the duodenal side, which may prevent drainage or cause infection due to content entrapment within an already diseased gallbladder. The curved region 608 can be arranged to use the natural anatomical pressure driven and gravitational based drainage to allow drainage out of the gallbladder whilst providing a more resistant path for reverse drainage or impaction from the duodenal region. This is more clearly illustrated in FIG. 7. For example, the bridging distance (i.e., the length of the cylindrical saddle region 606) may be lengthened to achieve a similar placement location and, as a consequences, provide further resistance to reverse drainage.

In some embodiments, stents 600a and/or 600b can include multiple curved regions 608, for example, to provide further resistance to backflow into gallbladder. Additionally, with some embodiments, the curved regions 608 may be imposed in more than one plane on a device, which again may offer better placement options, potential vessel rearrangement (thus reducing tension on device and reducing potential for migration) and/or reducing backflow pressure.

With some examples, the stents 600a and/or 600b can have a tapered body in conjunction with the curved regions 608 to provide a wide ingress aspect for drainage and a reduced egress aspect to prevent backflow pressure.

In some embodiments, the stents 600a and 600b can be manufactured using a curved mandrel and clamping sleeve.

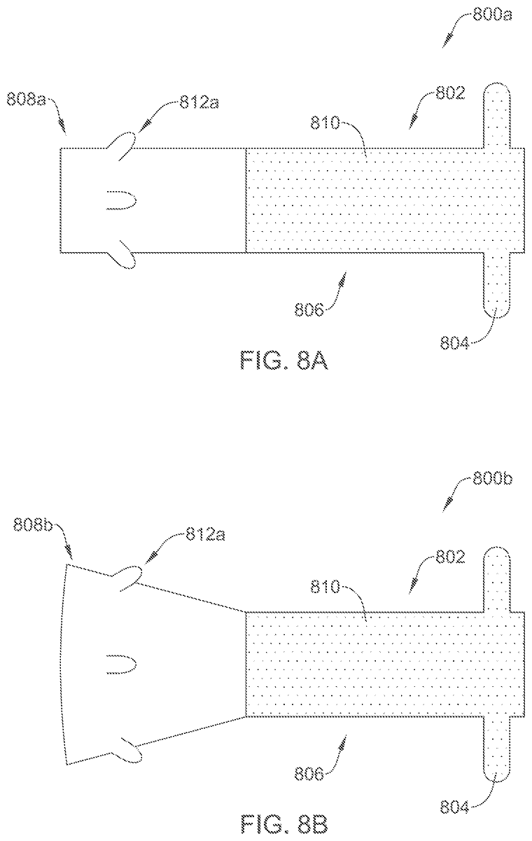

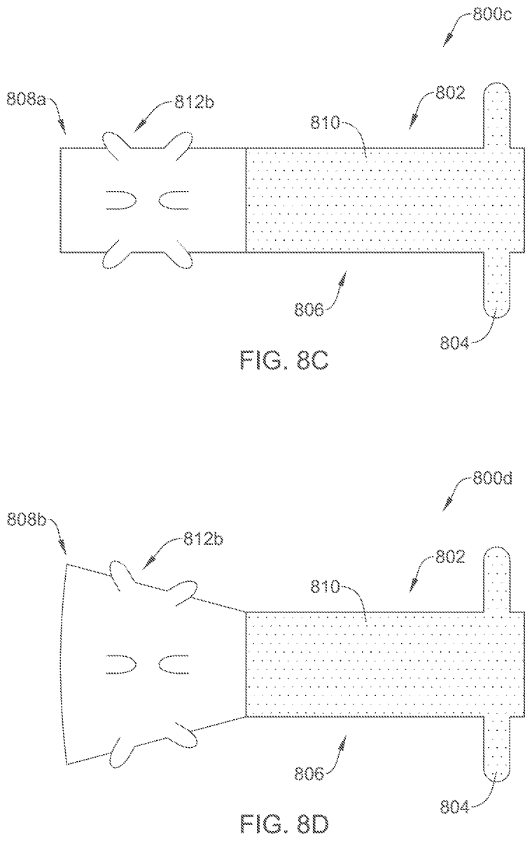

In some embodiments, stents, such as the Axios® stent, are used in EUS guided hepaticogastrostomy (HGS) treatment. FIG. 8A, FIG. 8B, FIG. 8C, and FIG. 8D illustrate stents 800a, 800b, 800c, and 800d, respectively. The stents 800a through 800d have a body 802, a flange 804 on the distal end and a straight or tapered proximal end 808a with a cylindrical saddle region 806 between the flange 804 and proximal end 808a. The distal end with the flange 804 can be arranged to be inserted downstream or toward the gastric section of the patient, while the proximal end 808a can be disposed upstream, or towards the hepatic section of the patient. The proximal end 808a can have a looped end to aid retrieval. Further, in some embodiments, the stents 800a through 800d can be bare (e.g., uncovered) or partially covered with covering 810 as shown. With some embodiments, covering 810 provides that the stents 800a through 800d can be used to bridge the gastric-hepatic drainage space, preventing leakage into the peritoneal space, while the bare end that is placed into the hepatic space allows for drainage and ingrowth for anti-migration.

The stents 800a through 800d further includes anti-migration fins (e.g., anti-migration fins 812a or anti-migration fins 812b). In general, the anti-migration fins can be loops upon the bare end (e.g., proximal end) of the stents, which protrude from the longitudinal plane of the stent. The anti-migration fins may be orientated facing either the hepatic direction or the distal direction or a mixture of both, as illustrated herein. The anti-migration fins augment the bare area of the stent in the hepatic area and provide a more acute anti-migration feature opposed to the bare surface. The bare surface by itself often takes a period (typically days to weeks depending on hepatic anatomy) to form a chronic anti-migration feature.

For example, FIG. 8A illustrates stent 800a, according to some embodiments of the present disclosure. The stent 800a has a straight proximal end 808a with anti-migration fins 812a facing downstream (e.g., away from the hepatic ducts).

FIG. 8B illustrates stent 800b, according to some embodiments of the present disclosure. The stent 800b has a tapered proximal end 808b with anti-migration fins 812a facing downstream like the stent 800a.

FIG. 8C illustrates stent 800c, according to some embodiments of the present disclosure. The stent 800c has a straight proximal end 808a with anti-migration fins 812b facing both upstream and downstream.

FIG. 8D illustrates stent 800d, according to some embodiments of the present disclosure. The stent 800d has a tapered proximal end 808b with anti-migration fins 812b facing both upstream and downstream.

As noted above, the present disclosure provides embodiments of stents, which can be used in an endoscopic retrograde cholangiopancreatography (ERCP) procedure. An ERCP procedure can include advancing an endoscope through the mouth and stomach and into the intestines. The endoscope can be advanced to an area of the intestines adjacent to the ampulla of Vater. A guidewire can be advanced from a working channel of the endoscope into the ampulla of Vater and into the common bile duct or pancreatic duct. A catheter carrying a self-expanding stent can be advanced over the guidewire to gain access to the common bile duct or the pancreatic duct. The catheter can retract a sheath to allow the self-expanding stent to expand. The sheath can be retracted partially to allow the first end or upstream end of the stent to expand within the common bile duct or pancreatic duct. After the upstream end has been deployed, the sheath can be further retracted to deploy the second or downstream end of the stent. The downstream end of the stent can be deployed in the ampulla of Vater, intestines, or other area of the common bile duct, or pancreatic duct. The cylindrical saddle region of the stent forms a fluid conduit or pathway between the common bile duct or pancreatic duct and the ampulla of Vater, intestines, or other area of the common bile duct, or pancreatic duct.

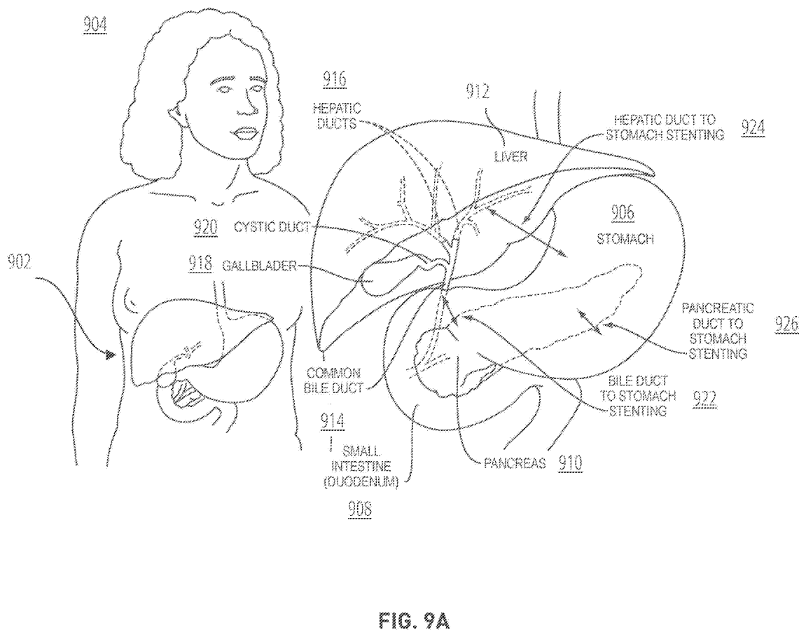

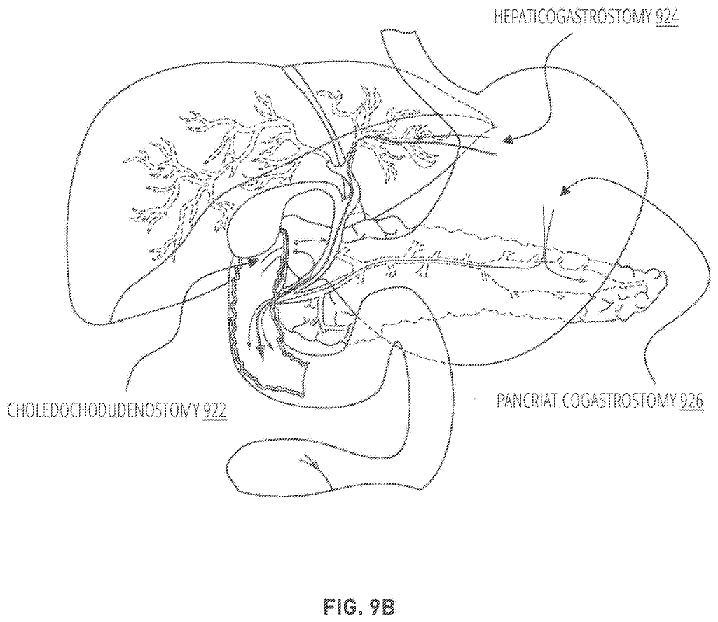

FIG. 9A and FIG. 9B illustrate examples of body lumens that can be connected by the stents disclosed herein. Areas within the abdominal cavity where stents described in this disclosure can be used to “span” or “connect” the common bile duct to the duodenum or the stomach to various positions in the biliary tree. Said differently, FIG. 9A and FIG. 9B illustrate various locations where stents can be placed within the abdominal cavity. In some embodiments, any of the stents disclosed herein can be placed in any of the locations illustrated in these figures. For example, any of the procedures illustrated in FIG. 9A or FIG. 9B can be used instead of an ERCP procedure. In some cases, an ERCP procedure can be unsuccessful or not possible, in those cases a stent can be placed through any of the pathways illustrated in FIG. 9A and FIG. 9B.

Turning more particularly to FIG. 9A, various locations within an abdominal cavity 902 of a patient 904 are depicted. For example, the stomach 906, duodenum 908, pancreas 910, liver 912, common bile duct 914, hepatic ducts 916, gallbladder 918, and cystic duct 920 are shown. Further, various stenting pathways are depicted.

For example, FIG. 9A and FIG. 9B depict a choledochodudenostomy 922, which connects the common bile duct 914 to the duodenum 908. For a choledochodudenostomy an endoscope can be advanced through the mouth and stomach 906 and into the duodenum 908. A target location in the common bile duct 914 can be identified using ultrasound guidance or other methods of guidance. A needle or catheter device can be advanced from the endoscope to puncture the wall of the duodenum 908 and the common bile duct 914. If a needle is used to access the common bile duct 914 then a guidewire can be placed with a catheter accessing the common bile duct 914 by advancing over the guidewire. The catheter can deploy a stent with an upstream end or flange within the common bile duct 914 and a downstream end or flange deployed in the duodenum 908 thereby forming a fluid conduit between the common bile duct 914 and the duodenum 908.

As another example, FIG. 9A and FIG. 9B depict a hepaticogastrostomy 924, which connects the hepatic cystic duct 920 to the stomach 906. To perform a hepaticogastrostomy 924, an endoscope can be advanced through the mouth and into the stomach 906. The target location in the liver 912 can be identified using ultrasound guidance or other methods of guidance. A needle or catheter device can be advanced to puncture the stomach 906 and liver 912. A guidewire can be placed in the liver 912 (after needle access) followed by advancing a catheter carrying a stent over the guidewire. An upstream end of the stent can be placed in the liver 912 and hepatic ducts 916 using the catheter. A downstream end of the stent is deployed within the stomach 906. The stent can have an uncovered portion on the end of the stent that is released inside the liver 912 and hepatic ducts 916. For example, the upstream end that is deployed within the liver 912 can have an uncovered portion of about 3-4 cm. The uncovered portion on the end of the stent can facilitate the flow of bile out of the liver and through the internal volume of the stent to drain to the stomach 906. The pressure in the liver 912 can assist the drainage of bile from the liver 912 through the stent and into the stomach 906. The downstream end of the stent deployed in the stomach 906 can be covered to reduce contact between the bile and the wall of the stomach 906.

In another example, FIG. 9A and FIG. 9A depict a pancriaticogastrostomy 926, in which an endoscope can be advanced through the mouth and into the stomach 906. A target location (e.g., duct) in the pancreas 910 can be identified using ultrasound guidance or other methods of guidance. A needle or catheter device can be advanced from the endoscope to puncture the wall of the stomach 906 and the duct in the pancreas 910. A guidewire can be placed in the duct of the pancreas 910 (after needle access) followed by advancing a catheter carrying a stent over the guidewire. An upstream end of the stent can be placed in the duct of the pancreas 910 using the catheter. A downstream end of the stent is deployed within the stomach 906 thereby forming a fluid conduit between the duct in the pancreas 910 and the stomach 906.

In some embodiments, the stents disclosed herein can be used to place a stent anterograde. Anterograde stent placement can be done in the common bile duct 914 and ducts of the pancreas 910. Anterograde stent placement is where the operator enters the upstream part of the common bile duct 914 (or a duct in the pancreas 910). The upstream part of the common bile duct 914 can be accessed percutaneously (e.g., transhepatic) or under EDS-guidance (e.g., transenteric targeting an intra- or extra-hepatic bile duct). After obtaining access to the upstream part of the bile duct, a guide wire is inserted and advanced downstream to cross the stricture and ampulla and advanced into the duodenum 908. A stent is then advanced anterogradely over the wire to cross the stricture and the ampulla until the downstream end of the stent is in the duodenum 908. The sheath is retracted relative to the stent to release the downstream flange or double-walled flange. The sheath and stent can then be retracted as a single unit until the flange abuts against the ampulla of Vater, signaled by the resistance encountered with retraction. The sheath is then retracted relative to the stent to deploy the upstream flange inside the common bile duct 914. A similar procedure can be used to place a stent anterograde in ducts in the pancreas 910 after obtaining upstream access to the pancreas 910.

It is noted that the above is not a complete description of exemplary procedures in which stents described herein can be employed. Instead, the above is given merely as an example and the above description should not be taken as limiting the scope of the disclosure, which is defined by the appended claims and the claims in any subsequent applications claiming priority hereto.

1. A stent comprising:
a body comprising an elongated tubular configuration and a foreshortened configuration, wherein an upstream end of the body expands into an upstream structure and the downstream end of the body expands into a flange structure when in the elongated tubular configuration, the body comprising a cylindrical saddle region disposed between the upstream structure and the flange structure, the body comprising a channel running helically around the body, the channel being defined on the exterior of the body and reflected on the interior of the body.

2. The stent of claim 1, wherein the channel has a consistent pitch along the longitudinal length of the body.

3. The stent of claim 1, wherein a pitch of the channel increases along a longitudinal length of the body.

4. The stent of claim 3, wherein the pitch increases along the longitudinal length of the body from the upstream end to the downstream end.

5. The stent of claim 1, wherein a width of the channel is wider at the upstream end of the body than at the downstream end of the body.

6. The stent of claim 1, wherein a width of the channel is wider at the downstream end of the body than at the upstream end of the body.

7. The stent of claim 1, wherein the upstream structure comprises a flange or a flare.

8. A stent comprising:
a body comprising an elongated tubular configuration and a foreshortened configuration, wherein an upstream end of the body expands into an upstream structure and the downstream end of the body expands into a flange structure when in the elongated tubular configuration, the body comprising a cylindrical saddle region disposed between the upstream structure and the flange structure, the body comprising a coating over the downstream end of the body and the cylindrical saddle region, the upstream structure comprising a plurality of anti-migration fins disposed on an external surface.

9. The stent of claim 8, the plurality of anti-migration fins comprising wire fins arranged to flare away from the upstream structure.

10. The stent of claim 8, the plurality of anti-migration fins comprising a point facing towards the downstream end of the body.

11. The stent of claim 8, at least one of the plurality of anti-migration fins comprising a point facing towards the downstream end of the body and at least one other one of the plurality of anti-migration fins comprising a point facing towards the upstream end of the body.

12. The stent of claim 8, the upstream structure comprising a flare.

13. A stent, comprising:
a body comprising an elongated tubular configuration and a foreshortened configuration, wherein an upstream end of the body expands into an upstream flange structure and the downstream end of the body expands into a downstream flange structure when in the elongated tubular configuration, the body comprising a cylindrical saddle region disposed between the upstream structure and the flange structure, the cylindrical saddle region comprising a curve along a longitudinal direction of the body.

14. The stent of claim 13, wherein the cylindrical saddle region comprises another curve along an axial direction of the body.

15. The stent of claim 13, wherein the curve is closer to the upstream flange structure than the downstream flange structure or closer to the downstream flange structure than the upstream flange structure.

16-20. (canceled)
